(19) United States
(12) Patent Application Publication
(10) Pub. No.: US 2022/0105225 A1
(43) Pub. Date: Apr. 7, 2022

(54) Smartphone Case With UV LED Sanitizer

(71) Applicant: Zhenwu Huang, Las Vegas, NV (US)

(72) Inventor: Zhenwu Huang, Las Vegas, NV (US)

(21) Appl. No.: 17/062,486

(22) Filed: Oct. 2, 2020

(51) Int. Cl. A61L 9/20; H04M 1/17; A45C 11/00; A45C 15/00

(52) U.S. Cl. CPC A61L 9/20; H04M 1/17; A45C 11/00; A45C 2011/002; A61L 2202/11; A61L 2202/23; A45C 15/00

(57) ABSTRACT

A protective smartphone case with UV LED sanitizer configured for attachment to a smartphone includes a plastic smartphone case body having a base, a top end, and a bottom end with four impact resistant sides which form the perimeter of the said smartphone case body; a back cover plate; a fixed bracket; a power connector; a touch switch module; a phone power socket; and an LED ultraviolet light module. Use of the protective smartphone case with UV LED sanitizer includes the steps of attaching a smartphone to the plastic smartphone case body, pointing the LED ultraviolet light module in the direction of the surface or object to be disinfected, positioning the LED ultraviolet light module at an optimal distance from the surface or object, engaging the touch switch module, irradiating the surface or object for a recommended period of time and disengaging the touch switch module.

### CROSS-REFERENCE TO RELATED APPLICATIONS

Not applicable.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### BACKGROUND OF THE INVENTION

### 1. Field of the Invention

The invention relates generally to the field of portable sanitizers which employ ultraviolet radiation. More specifically, the present invention relates to a smartphone case with a UV sanitizer configured to apply germicidal ultraviolet radiation to surfaces and objects.

### 2. Description of the Related Art

The world has an increased awareness of infectious disease and is currently searching for ways to combat the spread of pathogenic bacteria and viruses. Due to the COVID-19 pandemic, improved sanitizing devices are not only desired, they have become a necessary element in both living and work spaces.

It is now a well-known fact that COVID-19 is transmitted by human-to-human contact and is considered to be a highly infectious disease. One of the predominant mechanisms for pathogenic agents such as the novel coronavirus to be contagious is self-inoculation from contaminated surfaces. Self-inoculation could occur by poor hand hygiene or by not following common disease-controlling etiquettes such as frequent hand to face contact.

Contaminated objects and surfaces, sometimes referred to as fomites, are particularly hazardous when it comes to the spread of infectious disease. Surfaces may become contaminated by pathogens through coughing, sneezing and/or shedding by touch. Aerosolized pathogens will eventually settle on surfaces where they may infect others through contact. With dangerous pathogens such as SARS-CoV-2, the viral pathogen known to cause COVID-19, a person may innocently touch a contaminated surface and then proceed to touch vulnerable areas such as the face, nose or eye area and later become infected. In one study, SARS-CoV-2 was found to be viable on various surfaces from 4 to 72 hours under laboratory conditions. Other studies have shown that influenza viruses were still active on stainless steel 24 hours after contamination. Because certain viruses and bacteria may remain alive and infection on surfaces for a considerable amount of time, they may be spread among a vulnerable population in a short amount of time.

Germicidal techniques including the use of various types of sanitizers have been developed to combat the contamination of surfaces, and are well known in the art. Numerous methods inventions have been created and implemented to clean and sanitize both surfaces and the surrounding air. Among the most popular methods of sanitizing both surfaces and the surrounding air is the use of chemical sanitizers.

Chemical sanitizers for surfaces involve the introduction of a chemical mixture onto the surface. Chemical sanitizers ranging from alcohols to chlorine based sanitizers have been used for well over 100 years. In recent years, the use of pre-cut cloth towelettes soaked in a chemical solution has become immensely popular. Though this method of disinfecting air has proven effective, many chemical sanitizers can irritate respiratory and integumentary systems. Moreover, chemical sanitizers are not very portable and may be inconvenient to apply in many situations.

As an alternative to chemical sanitizers, the use of ultraviolet irradiation devices has become widely used. Ultraviolet sanitizing devices essentially consist of a device where ultraviolet lights irradiate surfaces and the surrounding space. Such ultraviolet irradiation has proven itself highly effective in killing pathogens, and several portable devices have been created. Ultraviolet radiation is mutagenic to bacteria, viruses and other microorganisms, with short wavelength ultraviolet radiation considered to be “germicidal” at wavelengths between 100-280 nanometers. Traditional ultraviolet sanitizer devices consisted of mercury vapor lamps which were bulky, toxic and energy intensive. The advent of light emitting diodes (LEDs) capable of emitting germicidal ultraviolet radiation has provided a new weapon in the fight against dangerous pathogens.

The use of smartphones has become ubiquitous in today's global and interconnected society. Smartphones are used in virtually every country in the world and have enabled users to be in communication with others in ways people from earlier generations could never have foreseen. The use of smartphones has extended beyond communications, though, with numerous applications available such as the use of cameras, lights, microphones and speakers.

Because smartphones are generally fragile and can suffer great damage through dropping or impacts, special cases have been developed to protect them. Today, most users of smartphones choose to protect their smartphone through the use of such a case. In general, a smartphone case consists of an impact resistant plastic capable of absorbing any sort of impact trauma which protects the smartphone itself. Many cases include features such as waterproof liners and moveable buttons which can be pressed to engage buttons on smartphones.

Presently, there exists a need for an improved, low maintenance, smartphone case which incorporates a UV LED sanitizer system and method for sanitizing surfaces. Such a system must be able to effectively replace chemical sanitizers. Additionally, such a system must be able to ensure that such a system is optimized to current health and safety standards. There currently exists a need for a smartphone case that is capable of providing germicidal ultraviolet radiation capable of disinfecting and sanitizing surfaces.

### SUMMARY

The present invention combines the function of a smartphone case and user operable UVC LED lights. The invention is easier to carry around than traditional chemical sanitizers and easier and safer to use. A smartphone offers a power supply for the UVC LED lights and adds new functions to a phone case. Not only does the invention offer protection for smart phones as an impact resistant and weatherproof case, but also offers a sanitizing means for different objects and surfaces.

At its essence, the smartphone case with UV LED sanitizer comprises a phone case body, a back cover plate, a fixed bracket, a power connector, a touch switch module, a phone power socket and an LED ultraviolet light module. The power socket of the smartphone acts as an external charging device that supplies power to the LED ultraviolet lamp module or to the mobile phone. It can also act as a connector between the mobile phone and external devices.

Such a system can effectively replace the use of chemical sanitizers for disinfecting surfaces and objects. Use of the smartphone case with UV LED sanitizer is conducive to individual compliance with current health and safety standards. Moreover, the smartphone case with UV LED sanitizer is capable of sanitizing an increased amount of any given surface while providing an easy-to-use system which may be networkable with other internet enabled devices.

An object of the present invention is to provide an improved UV sanitizing system which can be specifically configured to integrate with a plastic smartphone case and utilized to sanitize surfaces or objects. The invention is shaped and configured to look like a typical smartphone case, function as a protective case for typical smartphones, and provide germicidal UV radiation capable of sanitizing surfaces and objects.

Compared with other UV LED light sanitizer products, the present invention is not only capable of inhibiting the growth of pathogens such as bacteria and viruses, but also can be conveniently attached to a standard smartphone. As a method of operation, the UV disinfection can be carried performed with one hand. The smartphone case with UV LED sanitizer is easy to install and disassemble and is designed to withstand drops and other impact trauma. The smartphone case with UV LED sanitizer is also convenient for travelling and other situations where chemical sanitizers may not be allowed or available.

A further object of the invention is to provide a UV sanitizing system which is capable of sanitizing a greater surface area than existing UV LED sanitizer systems. The present invention consists of a plurality of LED units capable of emitting more intense germicidal radiation at greater distances and greater angles.

### BRIEF DESCRIPTION OF THE DRAWINGS

The invention directed by way of example, and not by way of limitation, in the figures of the accompanying drawings and in which like reference numerals refer to similar elements and in which:

FIG. 1 is a perspective illustration of an exemplary smartphone case with UV LED sanitizer in accordance with an embodiment of the invention;

FIG. 2 is an exploded perspective view of a smartphone case with UV LED sanitizer in accordance with an embodiment of the invention;

FIG. 3 is front elevation view of an exemplary smartphone case with UV LED sanitizer in accordance with an embodiment of the invention; and

FIG. 4 is a rear elevation view of an exemplary smartphone case with UV LED sanitizer in accordance with an embodiment of the invention; and

FIG. 5 is a perspective illustration of a smartphone being inserted into the exemplary smartphone case with UV LED sanitizer in accordance with an embodiment of the invention.

Unless otherwise indicated illustrations in the figures are not necessarily drawn to scale.

### DETAILED DESCRIPTION OF THE PREFERRED EMBODIMENTS

Terminology used herein is used for the purpose of describing particular embodiments only, and is not intended to limit the scope of the present invention. It must be understood that as used herein and in the appended claims, the singular forms “a,” “an,” and “the” include the plural reference unless the context clearly dictates otherwise. For example, a reference to “an element” is a reference to one or more elements and includes all equivalents known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Language that may be construed to express approximation should be so understood unless the context clearly dictates otherwise.

Unless defined otherwise, all technical and scientific terms used herein have the same meanings as commonly understood by a person of ordinary skill in the art to which this invention belongs. Preferred methods, techniques, devices, and materials are described. But any methods, techniques, devices, or materials similar or equivalent to those described herein may be used in the practice or testing of the present invention. Structures described herein should also be understood to refer to functional equivalents of such structures.

References to “one embodiment,” “one variant,” “an embodiment,” “a variant,” “various embodiments,” “numerous variants,” etc., may indicate that the embodiment(s) of the invention so described may include particular features, structures, or characteristics. However, not every embodiment or variant necessarily includes the particular features, structures, or characteristics. Further, repeated use of the phrase “in one embodiment,” or “in an exemplary embodiment,” or “a variant,” or “another variant,” do not necessarily refer to the same embodiment although they may. A description of an embodiment with several components in communication with each other does not imply that all such components are required. On the contrary, a variety of optional components are described to illustrate the wide variety of possible embodiments and/or variants of the present invention.

“Sanitizer” refers to a substance or device for killing or reducing levels of pathogenic microorganisms.

“Ultraviolet germicidal irradiation” or “UVGI” is a germicidal technique where ultraviolet radiation is used to kill or inactivate microorganisms. Ultraviolet radiation is mutagenic to bacteria, viruses and other microorganisms, with short-wavelength ultraviolet radiation considered to be “germicidal” at wavelengths between 100-280 nanometers.

“Software” may refer to prescribed rules and/or instructions used to operate a computer. Examples of software may include: Code segments in one or more computer-readable languages; graphical and or/textual instructions; applets; pre-compiled code; interpreted code; compiled code; and computer programs. An operating system or “OS” is software that manages computer hardware and software resources and provides common services for computer programs.

Certain embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages or other compilers, assemblers, interpreters or other computer languages or platforms.

A “smartphone” refers to a mobile device that combines cellular telephone and mobile computing functions into one unit. Smartphones are distinguishable from feature phones by their stronger hardware capabilities and extensive mobile operating systems, which facilitate wider software, internet access capabilites, and multimedia functionality alongside core phone functions such as voice calls and text messaging. Smartphones typically contain a number of metal-oxide-semiconductor (MOS) integrated circuit (IC) processor chips, include various sensors that can be leveraged by their software (such as a magnetometer, proximity sensors, barometer, gyroscope, or accelerometer), and support wireless communications protocols.

As is well known to those skilled in the art, many careful considerations and compromises typically must be made when designing the optimal manufacture or commercial implementation of such a smartphone case with UV LED sanitizer. A commercial implementation in accordance with the spirit and teachings of the invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art.

The exemplary smartphone case with UV LED sanitizer will now be described in detail with reference to embodiments thereof as illustrated in the accompanying drawings. Unless otherwise indicated illustrations in the figures are not necessarily drawn to scale.

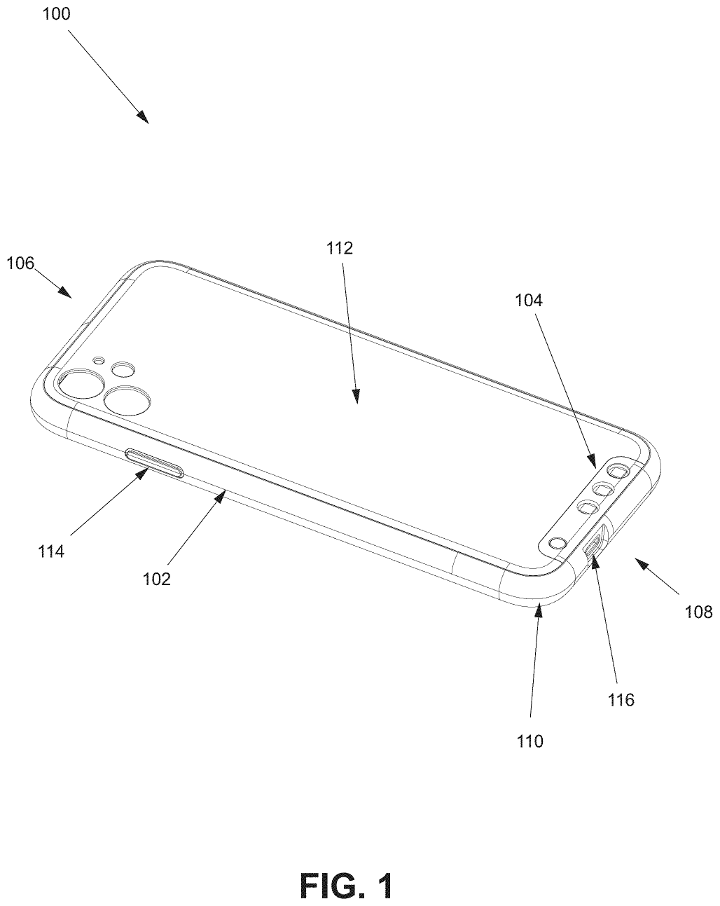

FIG. 1 is a perspective illustration of an exemplary smartphone case with UV LED sanitizer in accordance with an embodiment of the invention. The smartphone case with UV LED sanitizer 100 consists of two basic components, a plastic smartphone case body 102 and a LED ultraviolet lamp module 104. The phone case body 102 includes a top end 106 and a bottom end 108. The phone case body also includes four impact resistant sides 110 extending perpendicularly from the said base which form the perimeter of the said smartphone case body. The plastic smartphone case body 102 includes a cavity located at the bottom end of the said protective smartphone case with the cavity opening on the rear of the said protective smartphone case. The phone case body includes a rear plate 112. In this view, the rear plate 112 of the smartphone case with UV LED sanitizer is shown. In the preferred embodiment of the invention, the LED ultraviolet lamp module is positioned at the bottom end 108 of the rear of the plastic smartphone case. The LED ultraviolet light module 104 mounts inside the cavity in the bottom end of the said protective smartphone case with the fixed bracket providing a weatherproof protective seal.

The purpose of a plastic smartphone case body 102 is to protect the smartphone from impacts and other environmental hazards. The plastic smartphone case body 102 further protects the LED ultraviolet light module 104 as well. A plastic smartphone case body 102 can be molded or milled or shaped by numerous means known and appreciated in the art. Such a smartphone case body can be shaped to house different types of smartphones made by different manufacturers. Persons having skill in the art will further appreciate that there are numerous materials from which to choose in manufacturing a plastic smartphone case body 102. In some embodiments, a polycarbonate plastic can be used. In other embodiments, an acrylonitrile butadiene styrene (ABS) can be used. In other embodiments of the invention, a combination of polycarbonate and ABS plastics can be used. Other plastics and materials may also be used to manufacture such a plastic smartphone case body.

It will become readily apparent to persons having skill in the art that numerous accessories and functional features can be included or configured to be included in the manufacture of the plastic smartphone case body. Such features include, but are not limited to, buttons 114 used to engage or depress buttons located on smartphones. The plastic smartphone case body also includes an aperture for a power cord connection 116. Other features may include inner waterproof liners. Other features may include transparent smartphone face coverings. In embodiments of the invention, custom appearances such as different colors and graphics may be applied to the plastic smartphone case body 102 as well as the rear plate 112. In an embodiment of the invention, the rear plate 112 is simply affixed to the smartphone case body through the use of an adhesive such as cyanoacrylate or other suitable means. In another embodiment of the invention, the rear plate 112 may be attachable through plastic tabs or fasteners to provide for a customizable appearance.

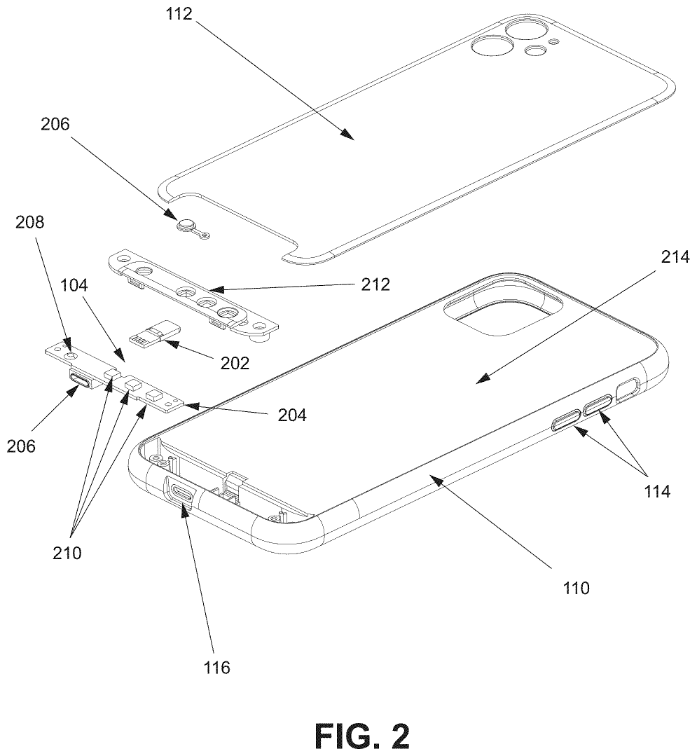

FIG. 2 is an exploded perspective view of a smartphone case with UV LED sanitizer in accordance with an embodiment of the invention. In an embodiment of the invention, the smartphone case with UV LED sanitizer 100 comprises the plastic smartphone case body 102, a back cover plate 112, a fixed bracket 212, a power connector 206, a touch switch module 208, a phone power socket 202 and an LED ultraviolet light module 104.

In an embodiment of the invention, the LED ultraviolet light module 104 consists of the touch switch module 208, the phone power socket 202, the power connector 206, and at least three LED light units 210. The touch switch module 208 and at least three LED light units 210 are mounted on a printed circuit board 204 to form a module. It will be readily understood by persons having skill in the art that an LED ultraviolet light module 104 may be configured and assembled by numerous techniques that are appreciated in the art. In embodiments of the invention, the voltage and current are provided through the power connector 206 or the phone power socket 202 and run through the printed circuit board 204 and UV LED lights 210. In alternative embodiments of the invention, the number of LED light units 210 can be greater than three. In other embodiments, the number of LED lights 210 can be less than three. It should be understood, though, that newer LED lights are smaller and are capable of being configured in such a manner so as to provide an array of LED UV lights capable of transmitting germicidal ultraviolet radiation at wider angles and greater distances. Persons having skill in the art will understand that a circuit is completed and the UV LED lights 210 transmit light by engaging the touch switch module 208.

In embodiments of the invention, the LED ultraviolet light module 104 is held in place and protected by a fixed bracket 212 mounted superior to the LED ultraviolet light module 104 and attaches to the plastic smartphone case. The fixed bracket 212 serves as a weatherproof protective seal which protects the LED ultraviolet light module 104 from the elements and other environmental hazards. In embodiments of the invention, LED ultraviolet light module 104 mounts inside a cavity located the bottom end of the protective smartphone case 102 with the fixed bracket 212 providing a weatherproof and protective seal. In one embodiment of the invention, the fixed bracket 212 is made from a plastic similar to that of the plastic smartphone case 102. In other embodiments, the fixed bracket 212 can be made from a metal or metal alloy.

In embodiments of the invention, a smartphone connects to the smartphone case through a phone power socket 202. In an embodiment of the invention, the phone power socket 202 of the exemplary smartphone case with UV LED sanitizer acts as an external connecting device that supplies power from a smartphone battery or circuits to the LED ultraviolet lamp module or to the mobile phone. In embodiments of the invention, a smartphone receives power or other signals through the power connector 206. The power connector 206 is configurable to receive standard types of phone charger and signal cables. It can also act as a connector between the mobile phone and external devices.

The use of next generation UV LED technology is an important factor in the present invention. Newer LED lights, sometimes referred to as Deep-UV LED lights, are smaller and are capable of emitting radiation of greater intensity. The development of Deep-UV LEDs has become particularly active. Furthermore, newer Deep-UV LED lights are capable of operating at voltages, currents and resistances used in typical smartphones. Moreover, LED lights are safer and more eco-friendly than traditional mercury vapor lamps originally used to generate germicidal UV radiation. In an embodiment of the invention, the LED lights 210 used transmit radiation in the UV-C band with a wavelength range between 200 and 280 nanometers. In one embodiment of the invention, the most intense radiation is transmitted at 275 nanometers. In alternative embodiments of the invention, different LED lights 210 can be employed to transmit different germicidal UV wavelength light. In one embodiment, the LED light module 104 operates at voltages and currents of 3.7V and 500 mA, with typical smartphone chargers and signal wires conducting at 5V and 500 mA. Persons having skill in the art will readily appreciate that voltages, currents and resistances may be adjusted through means and componets known and appreciated in the art to perfect embodiments of the invention. In one embodiment of the invention, the LED light module uses LED lights of the Qingdao Jason Electric Co. model JS35AG. However, other comparable and suitable LED light units can be used. In addition, the LED angles can be 30 degrees, 60 degrees, 90 degrees and 120 degrees. In the preferred embodiments of the invention, the smartphone case with UV LED sanitizer power connector 206 connects to power and signal sources via Universal Serial Bus (USB) connections. Non limiting examples of USB connections include, but are not limited to, USB-B and USB-C. In alternative embodiments of the invention, such voltages and currents may vary according to need.

The phone case body 102 includes a base 214 a top end 106 and a bottom end 108. The phone case body also includes four impact resistant sides 110 which form the outer perimeter of the smartphone case body. The four impact resistant sides extend perpendicularly from the base 214. The phone case body includes a rear plate 112. In this view, the rear plate 112 of the smartphone case with UV LED sanitizer is shown.

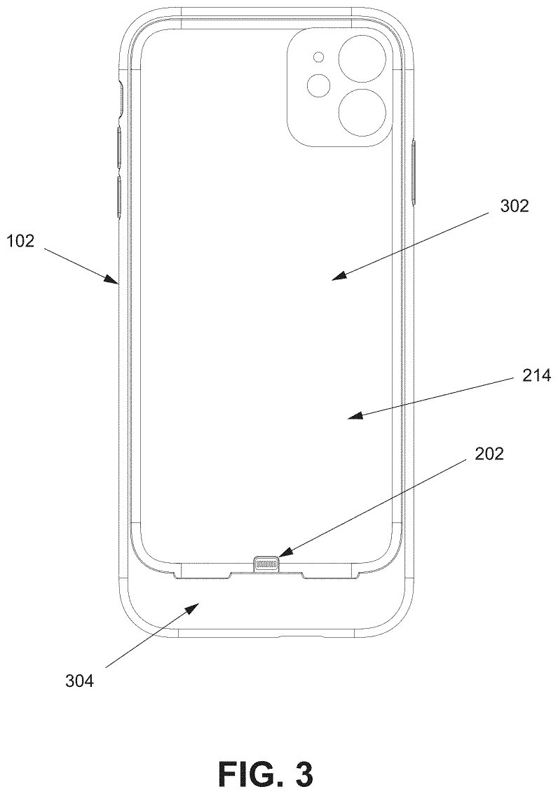

FIG. 3 is front elevation view of an exemplary smartphone case with UV LED sanitizer in accordance with an embodiment of the invention. In embodiments of the invention, a smartphone fits inside the plastic phone case body. The plastic phone case body 102 can be custom shaped having a phone cavity in the front side 302 and a LED ultraviolet light module cavity in the rear side 304. Persons having skill in the art will appreciate that a smartphone fits in this invention in the same way as any other ordinary phone cases. During installation, a user simply mates the phone power socket 202 to a smartphone USB port and then nestles the smartphone in the phone case body 102. Removal of the smartphone is the exact opposite (remove phone first and then disconnect the USB port).

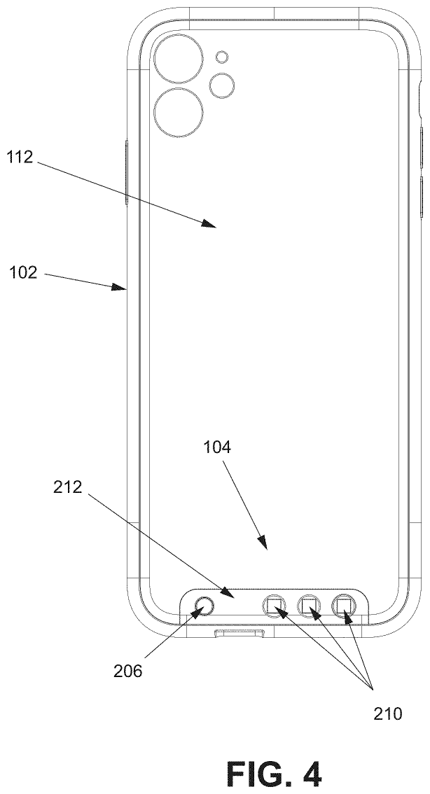

FIG. 4 is a rear elevation view of an exemplary smartphone case with UV LED sanitizer in accordance with an embodiment of the invention. In embodiments of the invention, the LED ultraviolet light module 104 is held in place and protected by a fixed bracket 212 mounted superior to the LED ultraviolet light module 104 and attaches to the plastic smartphone case. The fixed bracket 212 serves as a weatherproof protective seal which protects the LED ultraviolet light module 104 from the elements and other environmental hazards. In embodiments of the invention, LED ultraviolet light module 104 mounts inside a cavity located the bottom end of the protective smartphone case 102 with the fixed bracket 212 providing a weatherproof and protective seal. In some embodiments, further protective mechanism can be added to the fixed bracket 212 such as, but not limited to, transparent covers for the LED lights 210. In one embodiment of the invention, the fixed bracket 212 is made from a plastic similar to that of the plastic smartphone case 102. In other embodiments, the fixed bracket 212 can be made from a metal or metal alloy.

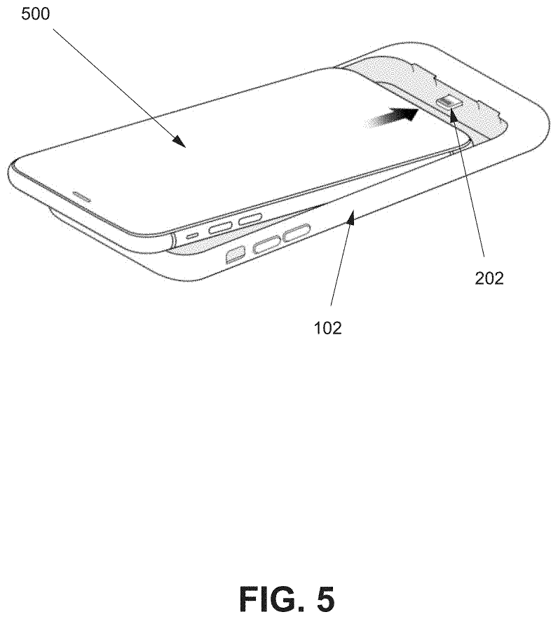

FIG. 5 is a perspective illustration of a smartphone being inserted into the exemplary smartphone case with UV LED sanitizer in accordance with an embodiment of the invention. A smartphone 500 fits in this invention in a similar way as smartphones fit inside any other ordinary phone case. During installation, though, a user mates the phone power socket 202 to a smartphone USB port and then nestles the smartphone in the phone case body 102. Removal of the smartphone is the exact opposite of attachment. A user may attach a power cord or USB cord to the power connector to charge a smartphone or to power the UV lights.

Use of the exemplary protective smartphone case with UV LED sanitizer is designed to be simple, yet highly effective. As a method of operation, the UV disinfection process can be carried performed with one hand. Use involves a user attaching a smartphone to the plastic smartphone case body. A user then simply points the LED ultraviolet light module in the direction of the surface or object to be disinfected. A user simultaneously positions the LED ultraviolet light module at an optimal distance from the surface or object to be disinfected. The user then engages the touch switch module which will turn on the UV LED units. In some embodiments of the invention, a user can engage the switch multiple times so as to prevent accidental operation. The user then irradiates the surface or object with germicidal ultraviolet radiation for a recommended period of time. Finally, a user disengages the touch switch module when finished.

Having fully described at least one embodiment of the smartphone case with UV LED sanitizer, other equivalent or alternative methods of implementing such an automated smartphone case with UV LED sanitizer according to the present invention will be apparent to those skilled in the art. Various aspects of the invention have been described above by way of illustration, and the specific embodiments disclosed are not intended to limit the invention to the particular forms disclosed. The particular implementation of the smartphone case with UV LED sanitizer may vary depending upon the particular context or application. By way of example, and not limitation, the system and method for the smartphone case with UV LED sanitizer described in the foregoing was principally directed to providing a UV LED sanitizer system for smartphone cases for various types of smartphones. However, similar techniques may instead be applied to cases for other handheld devices, which implementations of the present invention are contemplated as within the scope of the present invention. Additionally, software can be developed to power the UV LED sanitizer providing greater user functionality. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Although specific features of the smartphone case with UV LED sanitizerar is shown in some drawings and not others, persons skilled in the art will understand that this is for convenience. Each feature may be combined with any or all of the other features in accordance with the invention. The words “including,” “comprising,” “having,” and “with” as used herein are to be interpreted broadly and comprehensively, and are not limited to any physical interconnection. Claim elements and flowchart steps herein may have been numbered and/or lettered solely as an aid in readability and understanding. Any such numbering and lettering in itself is not intended to and should not be taken to indicate the ordering of elements and/or steps in the claims to be added at a later date.

Any amendment presented during the prosecution of the application for this patent is not a disclaimer of any claim element presented in the description or claims to be filed. Persons skilled in the art cannot reasonably be expected to draft a claim that would literally encompass each and every equivalent.

What is claimed is:

1. A protective smartphone case with UV LED sanitizer configured for attachment to a smartphone comprising:
a. a plastic smartphone case body having a base, a top end, and a bottom end with four impact resistant sides extending perpendicularly from the said base which form the perimeter of the said smartphone case body;
b. a back cover plate;
c. a fixed bracket;
d. a power connector;
e. a touch switch module;
f. a phone power socket; and
g. an LED ultraviolet light module.

2. The protective smartphone case of claim 1 wherein a smartphone fits inside the plastic smartphone case and connects with the phone power socket.

3. The protective smartphone case of claim 1 wherein the LED ultraviolet light module is powered by a smartphone battery connected to the phone power socket.

4. The protective smartphone case of claim 1 wherein a smartphone receives power or other signals through the power connector.

5. The protective smartphone case of claim 1 wherein the power connector is configurable to receive standard types of phone charger and signal cables.

6. The protective smartphone case of claim 1 wherein the LED ultraviolet light module consists of the touch switch module, the phone power socket, the power connector, and at least three LED light units, said touch switch module and at least three LED light units mounted on a printed circuit board.

7. The protective smartphone case of claim 6 wherein the LED ultraviolet light module consisting of the touch switch module, the phone power socket, the power connector, and at least three LED light units, said touch switch module and at least three LED light units mounted on a printed circuit board is protected by a fixed bracket mounted superior to the said LED ultraviolet light module and attaches to the plastic smartphone case.

8. The protective smartphone case of claim 1 wherein the plastic smartphone case body includes a cavity located at the bottom end of the said protective smartphone case with the cavity opening on the rear of the said protective smartphone case.

9. The protective smartphone case of claim 8 wherein the LED ultraviolet light module mounts inside the cavity in the bottom end of the said protective smartphone case with the fixed bracket providing a weatherproof protective seal.

10. The protective smartphone case of claim 6 wherein the at least three LED light units emit germicidal ultraviolet radiation at wavelengths ranging from 200 to 280 nanometers.

11. The protective smartphone case of claim 6 wherein the LED light units can be mounted at varying angles so as to provide a wider irradiation field.

12. A protective smartphone case configured for attachment to a smartphone comprising:
a. a custom shaped rectangular plastic smartphone case body having a phone cavity in the front side and a LED ultraviolet light module cavity in the rear side;
b. a back cover plate;
c. a fixed bracket;
d. a power connector;
e. a touch switch module;
f. a phone power socket; and
g. an LED ultraviolet light module.

13. The protective smartphone case of claim 12 wherein a smartphone fits inside the phone cavity of the custom shaped rectangular plastic smartphone case and connects with the phone power socket.

14. The protective smartphone case of claim 12 wherein the LED ultraviolet light module is powered by a smartphone battery connected to the phone power socket.

15. The protective smartphone case of claim 12 wherein a smartphone receives power or other signals through the power connector.

16. The protective smartphone case of claim 12 wherein the power connector is configurable to receive standard types of phone charger and signal cables.

17. The protective smartphone case of claim 12 wherein the LED ultraviolet light module consists of the touch switch module, the phone power socket, the power connector, and at least three LED light units, said touch switch module and at least three LED light units mounted on a printed circuit board, the said LED ultraviolet light module configured to emit germicidal ultraviolet radiation at wavelengths ranging from 200 to 280 nanometers.

18. The improved protective smartphone case of claim 17 wherein the LED ultraviolet light module consisting of the touch switch module, the phone power socket, the power connector, and at least three LED light units, said touch switch module and at least three LED light units mounted on a printed circuit board is protected by a fixed bracket mounted superior to the said LED ultraviolet light module and attaches to the plastic smartphone case.

19. The protective smartphone case of claim 12 wherein the LED ultraviolet light module is mounted inside the LED ultraviolet light module cavity of the custom shaped rectangular plastic smartphone case body with the fixed bracket providing a weatherproof protective seal.

20. A method of disinfecting surfaces and objects with a UV LED sanitizer apparatus configured for attachment to a smartphone, said apparatus comprising:
a. a plastic smartphone case body having a base, a top end, and a bottom end with four impact resistant sides extending perpendicularly from the said base which form the perimeter of the said smartphone case body;
b. a back cover plate;
c. a power connector;
d. a fixed bracket;
e. a touch switch module;
f. a phone power socket; and
g. an LED ultraviolet light module;
said method comprising the steps of:
1) attaching a smartphone to the plastic smartphone case body;
2) pointing the LED ultraviolet light module in the direction of the surface or object to be disinfected;
3) positioning the LED ultraviolet light module at an optimal distance from the surface or object to be disinfected;
4) engaging the touch switch module;
5) irradiating the surface or object with germicidal ultraviolet radiation for a recommended period of time; and
6) disengaging the touch switch module.
